(19) United States
(12) Patent Application Publication
(10) Pub. No.: US 2024/0131039 A1
(43) Pub. Date: Apr. 25, 2024

(54) TREATMENT OF GASTROINTESTINAL DISORDERS

(71) Applicant: Greenspace Labs, Inc., Dover, DE (US)

(72) Inventors: MCLAREN DUNCAN, Encinitas, CA (US); WILLIAM ALBRO, JR., Middletown, RI (US)

(21) Appl. No.: 18/490,538

(22) Filed: Oct. 18, 2023

Related U.S. Application Data
(60) Provisional application No. 63/380,536, filed on Oct. 21, 2022.

(51) Int. Cl. A61K 31/00; A61K 9/20; A61K 9/28

(52) U.S. Cl. CPC A61K 31/658; A61K 9/2009; A61K 9/2013; A61K 9/2018; A61K 9/2027; A61K 9/2054; A61K 9/2846

(57) ABSTRACT

The invention relates to compositions and formulations for the treatment of disorders of the gastrointestinal (GI) tract. Also provided are methods for treating, preventing, and/or alleviating disorders of the GI tract.

### CLAIM OF PRIORITY UNDER 35 U.S.C. § 119

The present Application for Patent claims benefit of Provisional Application No. 63/380,536 entitled “TREATMENT OF GASTROINTESTINAL DISORDERS” filed Oct. 21, 2022, and hereby expressly incorporated by reference herein.

### BACKGROUND

### Field

Provided herein are compositions and formulations suitable for the treatment of disorders of the gastrointestinal (GI) tract. Also provided are methods for treating, preventing, and/or alleviating disorders of the gastrointestinal tract.

### Background

Irritable bowel syndrome (IBS) affects about 15% of the U.S. population. There are still no effective and safe medications approved for the treatment of abdominal pain associated with bowel symptoms in IBS.

### SUMMARY

Embodiments of the invention relate to a composition for preventing or alleviating a disorder of the gastrointestinal tract (GI). The the composition can include at least two active agents selected from a cannabinoid and/or a terpene. The active agent can be CBD, CBG, CBDA, THC, caryophyllene, limonene, linalool, alpha pinene, myrcene, humulene, and/or the like. In some embodiments, the two active agents act synergistically.

Some embodiments of the invention relate to a formulation including any composition described herein.

In some embodiments, the formulation can be a capsule or tablet. In some embodiments, the formulation can be a coated capsule or tablet.

In some embodiments, the formulation can include microcyrstalline cellulose, croscarmellose sodium, magnesium stereate, and/or colloidal silicon dioxide. In some embodiments, the formulation can further include mannitol, and/or sodium stearyl.

In some embodiments, the gastrointestinal disorder can be inflammatory bowel syndrome (IBS), gastrointestinal motility disorders, functional gastrointestinal disorders, gastroesophageal reflux disease (GERD), duodenogastric reflux, Crohn's disease, ulcerative colitis, inflammatory bowel disease (IBD), functional heartburn, dyspepsia, functional dyspepsia, nonulcer dyspepsia, gastroparesis, chronic intestinal pseudo-obstruction, colonic pseudo-obstruction, and/or the like.

Some embodiments of the invention relate to a method for preventing or alleviating a disorder of the gastrointestinal tract. The method can include administering a composition or formulation disclosed herein to a subject with a gastrointestinal disorder in a dosage amount sufficient to prevent or alleviate a disorder of the gastrointestinal tract. In some embodiments, the gastrointestinal disorder is IBS, gastrointestinal motility disorders; functional gastrointestinal disorders, GERD, duodenogastric reflux, Crohn's disease, ulcerative colitis, inflammatory bowel disease (IBD), functional heartburn, dyspepsia, functional dyspepsia, nonulcer dyspepsia, gastroparesis, chronic intestinal pseudo-obstruction, colonic pseudo-obstruction, and/or the like. In some embodiments, the administration is by oral administration.

### BRIEF DESCRIPTION OF THE DRAWINGS

FIG. 1A depicts results from experiments predicting potential interaction/miscibility between the API and polymers that can be used in formulations comprising CBD (miscibility modeling).

FIG. 1B depicts results from experiments predicting potential interaction/miscibility between the API and polymers that can be used in formulations comprising CBGA (miscibility modeling).

FIG. 1C depicts results from experiments predicting potential interaction/miscibility between the API and polymers that can be used in formulations comprising CBDA (miscibility modeling).

FIG. 2 depicts results from microevaporating screening experiments testing re-dissolution of formulations of the invention comprising CBD (microevaporative screening with polymers and surfactants).

FIG. 3 depicts results from microevaporating screening experiments testing re-dissolution of formulations of the invention comprising CBGA (microevaporative screening with polymers and surfactants).

FIG. 4 depicts results from microevaporating screening experiments testing re-dissolution of formulations of the invention comprising CBDA (microevaporative screening with polymers and surfactants).

FIG. 5A depicts results from experiments testing non-sink dissolution of formulations of the invention comprising CBD,

FIG. 5B depicts results from experiments testing non-sink dissolution of formulations of the invention comprising CBGA.

FIG. 5C depicts results from experiments testing non-sink dissolution of formulations of the invention comprising CBDA.

### DETAILED DESCRIPTION

Provided in certain embodiments herein are methods for and compositions useful for preventing or alleviating disorders of the gastrointestinal tract or symptoms thereof in an individual comprising orally administering to said individual a composition comprising at least one active agent capable of preventing or alleviating gastrointestinal disorders.

### Compositions

Some embodiments of the invention are related to a composition useful for preventing or alleviating disorders of the gastrointestinal tract or symptoms thereof. In some embodiments, the composition can include but is not limited to 1, 2, 3, 4, or more active agents. The active agent can include but is not limited to having an effect of preventing or alleviating disorders of the gastrointestinal tract, for example increased gastrointestinal motility, decreased inflammation, decreased gastrointestinal pain, decreased visceral pain, and/or the like. Specific effects can include improvement in abdominal bloating and distention, improvement in abdominal cramping, improvement in nausea, improvement in diarrhea, and/or the like. In some embodiments, the active agents can have a synergistic effect. In some embodiments, an active agent can have a synergistic effect with a non-active agent. A non-active agent can be defined as any ingredient used in the formulation that may or may not have an effect when used alone.

The active agent can be at least one of a cannabinoid or a terpene. Exemplary cannabinoids can include, but are not limited to tetrahydrocannabinolic acid A (THCA-A), tetrahydrocannabinolic acid B (THCAB), tetrahydrocannabinol (THC), tetrahydrocannabinolic acid C (THCA-C), tetrahydrocannabinol C (THC-C), tetrahydrocannabivarinic acid (THCVA), tetrahydrocannabivarin (THCV), tetrahydrocannabiorcolic acid (THCA-C), tetrahydrocannabiorcol (THC-C), delta-7-cis-iso-tetrahydrocannabivarin, delta-8-tetrahydrocannabinolic acid (Δ8-THCA), delta-9-tetrahydrocannabinol (Δ9-THC), cannabidiolic acid (CBDA), cannabidiol (CBD), cannabidiol monomethylether (CBDM), cannabidiol-C(CBD-C), cannabidivarinic acid (CBDVA), cannabidivarin (CBDV), cannabidiorcol (CBD-C), cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabivarin (CBV), cannabinol-C(CBN-C), cannabiorcol (CBN-C1), cannabinodiol (CBND), cannabinodivarin (CBVD), cannabitriol (CBT), 10-Ethoxy-9-hydroxy-delta-6a-tetrahydrocannabinol, 8,9-dihydroxy-delta-6a-tetrahydrocannabinol (8,9-Di-OH-CBT-C5), cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (Δ9-cis-THC), cannabiripsol (CBR), -3,4,5,6-tetrahydro-7-hydroxy-alpha-alpha-2-trimethyl-9-n-propyl-2,6-methano-2H-1-benzoxocin-5-methanol (OH-iso-HHCV), trihydroxy-delta-9-tetrahydrocannabinol (triOH-THC), an isocanabinoid, any other cannabinoid, and any combination thereof.

Exemplary terpenes can include, but are not limited to myrcene, limonene, linalool, beta-caryophyllene, alpha-pinene and beta-pinene, alpha-bisabolol, eucalyptol, trans-nerolidol, humulene, delta-3-carene, camphene, borneol, terpineol, valencene, geraniol, eugenol, sabinene, phellandrene, borneol, isoborneol, phytol, menthol, geraniol, citronellol, ocimene, halomon, thymol, carvacrol, thujene, camphene, camphor, verbenone, botrydial, ngaione, cuparane, labdane, ferruginol, cafestol, any other terpene, and any combination thereof.

In some embodiments, the composition or formulation can include less than 1, 0.8, 0.6, 0.4., 0.3, 0.1% w/w THC.

In specific embodiments, the composition can include one, or a plurality, or all of: CBD, CBG, CBDA, THC, caryophyllene, limonene, linalool, alpha pinene, myrcene, humulene, and/or the like.

In some embodiments, the ratio of CBD to CBG can be less than 1:1, 1:1, 2:1, 3:1, 4:1, 5:1 or more.

In some embodiments, the ratio of CBD to CBDA can be less than 1:1, 1:1, 2:1, 3:1, 4:1, 5:1 or more.

In some embodiments, the ratio of CBG to CBDA can be less than 1:1, 1:1, 2:1, 3:1, 4:1, 5:1 or more.

In a specific embodiment, the composition can include a 3:1:1 ratio of 15 mg CBD, 5 mg CBG, 5 mg CBDA.

Experiments in the Examples were completed using CBGA. Since CBGA is a precursor to CBG, it would be understood that an effect produced by a formulation containing CBGA, would be expected of a formulation where the CBGA is replaced with CBG.

In a specific embodiment, the composition can include a ratio of caryophyllene 3 mg, limonene 3 mg, linalool 1.5 mg, alpha pinene: 2 mg, myrcene 0.25 mg, humulene 1 mg.

Terpene concentrations in dried cannabis flowers typically range from 1% to 3% by weight, but this can vary. For example, a strain rich in myrcene can have 1-2% of myrcene by weight. This translates to 10-20 mg of myrcene per gram of dried flower.

In formulated products, terpene concentrations can vary even more, especially if terpenes are added back into the product after extraction. For a vape liquid or oil, terpene concentrations can range from, for example, 1% to 5% or more of the total volume, but this can be higher or lower depending on the product. For example, in a 1 mL vape cartridge containing 5% terpenes, there would be 50 mg of total terpenes.

When using terpenes in formulation, they can be obtained as concentrated isolates or blends. These can be diluted before being added to products. A typical dilution can involve adding terpenes at a 1-5% concentration, but this can vary based on the product type and desired effect.

In various embodiments of the invention that vary from the formulations discussed above, other cannabinoids can be substituted for CBD, CBG and/or CBDA. Likewise, other terpenes can be substituted for caryophyllene, limonene, linalool, alpha pinene, myrcene, and/or humulene.

“Compound derived from a Cannabis plant,” as used herein, can be defined as a compound naturally found in Cannabis. The actual compound used in the composition that is naturally found in Cannabis can be produced from another source.

The active agent can be natural or synthetic. Natural therapeutic agents can be prepared from compounds found occurring in nature, which contain active components in extract form created from sources, including plants, microbes, minerals and animals (see for example, Mathur, S., & Mathur, S. (2017). Drug development: Lessons from nature (Review). Biomedical Reports, 6, 612-614. https://doi.org/10.3892/br.2017.909), which is fully incorporated by references herein. A synthetic substance or synthetic compound can refer to a substance that is man-made by synthesis, rather than being produced by nature. It can also refer to a substance or compound formed under human control by any chemical reaction, either by chemical synthesis (chemosynthesis) or by biosynthesis.

In some embodiments, the composition or formulation including the composition can include about 1%, 5%, 10%, 15%, 20%, 25%, 30%, 35%, 40%, 45%, 50%, 55%, 60%, 65%, 70%, 75%, 80%, 85%, 90% w/w or more cannabinoid(s).

In some embodiments, the composition or formulation including the composition can include about 0.001%, 0.01%, 0.1%, 1%, 5%, 10%, 15%, 20%, 25%, 30%, 35%, 40%, 45%, 50%, 55%, 60%, 65%, 70%, 75%, 80%, 85%, 90% w/w or more terpenoid(s) or terpene(s).

Where there are at least two active agents of the composition, or formulation including the composition, the two active agents together can have synergistic effects as provided in Table 1. For example, formula 1 includes a combination of CBG and CBD which exhibits synergistic effects. For example, formula 9 includes a combination of a terpene and CBG which exhibits synergistic effects.

Such synergistic effects can be likened to the “entourage effect” associated with Cannabis use that involves the synergistic interaction of multiple active components of the flower with receptors throughout the body. With this effect, the mixture of components demonstrates greater therapeutic efficacy than any of its constituent compounds in isolation.

The use of the terms, “synergistic” and “synergistically effective,” are used in the present invention to mean a biological effect created from the application of two or more agents to produce a biological effect that is greater than the sum of the biological effects produced by the application of the individual agents. Quantification of synergistic effects can be found in or adapted from S. R. Colby, “Calculating Synergistic and Antagonistic Response of Herbicide Combinations” Weeds 15(1): 20-23, 1967; the entire contents of the foregoing is fully incorporated by reference herein.

TABLE 1
Synergistic combinations of Formulations

| | | | | Non- | | |
|---|---|---|---|---|---|---|
| | | | | CBD/CBG/ | | |
| | | | | CBDA | | |
| | CBD | CBG | CBDA | Cannabinoid A | Terpene A | Terpene B |
| CBD | | Formula 1 | Formula 2 | Formula 3 | Formula 4 | Formula 5 |
| CBG | Formula 6 | | Formula 7 | Formula 8 | Formula 9 | Formula 10 |
| CBDA | Formula 11 | Formula 12 | | Formula 14 | Formula 15 | Formula 16 |
| Non- | Formula 17 | Formula 18 | Formula 19 | Formula 20 | Formula 21 | Formula 22 |
| CBD/CBG/ | | | | | | |
| CBDA | | | | | | |
| Cannabinoid A | | | | | | |
| Terpene 1 | Formula 23 | Formula 24 | Formula 25 | Formula 26 | Formula 27 | Formula 28 |
| Terpene 2 | Formula 29 | Formula 30 | Formula 31 | Formula 32 | Formula 33 | Formula 34 |

In some embodiments, the present invention provides pharmaceutical compositions including a pharmaceutically acceptable excipient along with a therapeutically effective amount of the agents described herein. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

In some embodiments, the pharmaceutical compositions according to the invention can be formulated for delivery via any route of administration. “Route of administration” can refer to any administration pathway known in the art, including but not limited to aerosol, nasal, oral, transmucosal, transdermal or parenteral. “Transdermal” administration can be accomplished using a topical cream or ointment or by means of a transdermal patch. Via the topical route, the pharmaceutical compositions based on compounds according to the invention can be formulated for treating the skin and mucous membranes and can be in the form of ointments, creams, milks, salves, powders, impregnated pads, solutions, gels, sprays, lotions, or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions can be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions can be in the form of solutions or suspensions for infusion or for injection. The compositions can also be administered by vaporization or the like.

In some embodiments, the formulation can be in the form of a powder, granules, micropellets, nanopellets, microparticles, nanoparticles, a tablet, an effervescent tablet, a melting tablet, a disintegrating tablet, an orally disintegrating tablet, a foam, a gel, a solid solution, an emulsion, a liquid or semi-liquid solution, a gum, a wafer (e.g., dissolving or disintegrating), capsule (e.g., dissolving or disintegrating), or a combination thereof. In certain embodiments, the composition can be in the form of a film, a patch, a lozenge, or the like.

In some embodiments, the formulation can be engineered to deliver an active pharmaceutical ingredient (API) at target locations in the GI tract.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier can be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it can come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted, or prepared in an emulsion or syrup for oral administration. Pharmaceutically acceptable solid or liquid carriers can be added to enhance or stabilize the composition, or to facilitate preparation of the composition. Liquid carriers can include but are not limited to syrup, peanut oil, olive oil, glycerin, saline, alcohols and water. Solid carriers can include but are not limited to starch, lactose, calcium sulfate, dihydrate, terra alba, magnesium stearate or stearic acid, talc, pectin, acacia, agar or gelatin. The carrier can also include a sustained release material such as glyceryl monostearate or glyceryl distearate, alone or with a wax.

The pharmaceutical preparations are made following the conventional techniques of pharmacy and can include but are not limited to milling, mixing, granulation, and compressing, when necessary, for tablet forms; or milling, mixing, and filling for hard gelatin capsule forms. When a liquid carrier is used, the preparation will be in the form of a syrup, elixir, emulsion, or an aqueous or non-aqueous suspension. Such a liquid formulation can be administered directly by mouth (p.o.) or filled into a soft gelatin capsule.

The pharmaceutical compositions according to the invention can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly.

In some embodiments, the compositions and formulations described herein can be suitable for oral administration and/or are administered orally, in a manner that delivers the composition, or an active agent contained therein, to a gastrointestinal surface. In some embodiments, an orally administered composition can be delivered by an orally disintegrating or dissolving formulation, or a powder formulation. In certain embodiments, liquid oral compositions (e.g., suspensions) can be utilized in any of the methods described herein.

In some embodiments, the formulation can include a stabilizing agent (e.g., a polymer). Examples of suitable stabilizing agents can include, but are not limited to, Kollidon va 64 (vinylpyrrolidone-vinyl acetate copolymers), Soluplus (polyvinyl caprolactam-polyvinyl acetate-polyethylene glycol graft co-poly-mer), Eudragit epo (cationic copolymer made from methyl methacrylate, dimethylaminoethyl methacrylate, and butyl methacrylate), ethyl cellulose, Poloxamer 188 (co-polymers of polytheylene oxide and polypropylene oxide), Eudragit s100 (methacrylic acid and methyl methacrylate copolymer), Eudragit 1100 (methacrylic acid and methyl methacrylate copolymer), Eudragit 1100-55 (methacrylic acid and ethyl acrylate copolymer), polyethylene glycol 1500, polyethylene glycol 6000, acacia, agar, albumin, alginic acid, aluminum stearate, ammoniurn alginate, ascorbic acid, ascorbyl palmitate, bentonite, butylated hydroxytoluene, calcium alginate, calcium stearate, carboxymethylcellulose calcium, carboxymethylcellulose sodium, carrageenan, cellulose, microcrystalline and carboxymethylcellulose sodium, ceratonia, colloidal silicon dioxide, cyclodextrins, diethanolamine, edetates, ethylcellulose, ethylene glycol palmitostearate, glycerin monostearate, guar gum, hectorite, hydroxpropyl betadex, hydroxypropyl cellulose. hypromellose, inulin, invert sugar, lauric acid, lecithin, magnesium aluminumn silicate, mineral oil and lanolin alcohols, monoethanolamine, pectin, pentetic acid, phospholipids, polacrilin potassium, poloxamer, polyvinyl alcohol, potassium alginate, potassium chloride, povidone, propyl gallate, propylene glycol, propylene glycol alginate, raffinose, sodium acetate, sodium alginate, sodium borate, sodium stearyl fumarate, sorbitol, stearyl alcohol, sulfobutylether b-cyclodextrin, tagatose, trehalose, triethanolanine, white wax, xanthan gum, xylitol, yellow wax, zinc acetate, and/or the like.

In some embodiments, the formulation can include a solubilizing agent. Solubilizing agents can also be referred to as plasticizers. Examples of suitable solubilizing agents can include, but are not limited to, sodium lauryl sulfate, polysorbate 80, polyethyleglycol (15)-hydroxystearate, anionic emulsifying wax, cetylpyridinium chloride, cyclodextrins, glycerin monostearate, lecithin, meglumine, nonionic emulsifying wax, phospholipids, poloxamer, polyoxyethylene alkyl ethers, polyoxyethylene castor oil derivatives, polyoxyethylene sorbitan fatty acid esters, polyoxyethylene stearates, polyoxylglycerides, sorbitan esters, stearic acid, sulfobutylether b-cyclodextrin, tricaprylin, triolein, vitamin E polyethylene glycol succinate, and/or the like.

In some embodiments, the formulation can include a filler. Examples of suitable fillers can include, but are not limited to, mannitol M100, microcrystalline cellulose PH 101, and/or the like.

In some embodiments, the formulation can include a disintegrant. Examples of suitable disintegrants can include, but are not limited to hydroxypropyl starch, lactose, monohydrate and corn starch, alginic acid, calcium alginate, carboxymethylcellulose calcium, carboxymethylcellulose sodium, powdered cellulose, chitosan, colloidal silicon dioxide, corn starch and pregelatinized starch, 200 croscarmellose sodium, crospovidone, docusate sodium, glycine, guar gum, hydroxypropyl cellulose, low-substituted, magnesium aluminum silicate, methylcellulose, microcrystalline cellulose, polacrilin potassium, povidone, sodium alginate, sodium starch glycolate, starch, pregelatinized, and/or the like.

In some embodiments, the formulation can include a lubricant. Examples of suitable lubricants can include, but are not limited to, magnesium stearate, calcium stearate, glycerin monostearate, glyceryl behenate, glyceryl palmitostearate, hydrogenated castor oil, hydrogenated vegetable oil type I, light mineral oil, magnesium lauryl sulfate, medium-chain triglycerides, mineral oil, myristic acid, palmitic acid, poloxamer, polyethylene glycol, sodium benzoate, sodium lauryl sulfate, sodium stearyl fumarate, stearic acid, talc, zinc stearate, and/or the like.

In some embodiments, the formulation can include a glidant. Examples of suitable glidants can include, but are not limited to calcium phosphate—tribasic, powdered cellulose, colloidal silicon dioxide, hydrophobic colloidal silica, magnesium oxide, magnesium silicate, magnesium trisilicate, silicon dioxide, talc, and/or the like.

In some embodiments, for example, in coated tablet formulations, the formulation can include an enteric coating polymer. Examples of suitable enteric coating polymers can include, but are not limited to, acetyltributyl citrate, carborners, cellulose acetate phthalate, colonic drug delivery, guar gum, hypromellose acetate succinate, hypromellose phthalate, polymethacrylates, polyvinyl acetate phthalate, shellac, polyvinyl acetate phthalate (phthalavin enteric coating polymer, PVAP), tributyl citrate, triethyl citrate, triolein, white wax, zein, and/or the like.

In some embodiments, for example, in coated tablet formulations, the formulation can include an anti-tacking agent. Examples of suitable anti-tacking agents can include, but are not limited to, calcium phosphate, tribasic, calcium silicate, colloidal silicon dioxide, hydrophobic colloidal silica, leucine, magnesium oxide, magnesium silicate, magnesium trisilicate, tale, triethyl citrate, mono and di-glycerides and/or the like.

In some embodiments, the formulation can be in the form of a spray dried dispersion (SDD). The spray drying process consists of dissolving a drug and polymer in an organic solvent and spraying it with a stream of heated nitrogen gas to remove the organic solvent.

The dried powder is separated from the gas by a cyclone or filter. Spray drying creates dried particles that can be further processed into solid oral dosage forms with enhanced bioavailability. The spray dried dispersion can include the composition including one or more active agents and a stabilizing agent. In some embodiments, the dispersion can include a solubilizing agent. For example, the spray dried dispersion can include 1-25% w/w active agents, 25-95% w/w stabilizing agent, and 0-10% w/w solubilizing agent. For example, the SDD can include 1, 5, 10, 15, 20, 25% w/w or more active agent(s). For example, the SDD can include 25, 30, 35, 40, 45, 50, 55, 60, 65, 70, 75, 80, 85, 90, 95% w/w or more stabilizing agent(s). For example, the SDD can include 0.5, 1, 2, 3, 4, 5, 6, 7, 8, 9, 10% w/w or more solubilizing agent(s).

In some embodiments, the formulation is in the form of an amorphous solid dispersion (ASD). ASDs can include an amorphous active pharmaceutical ingredient (API) dispersed in a polymer matrix. Polymers in ASDs can function by disarranging the crystalline lattice of the API and produce a higher energy amorphous state which exhibits higher dissolution rate, solubility, and bioavailability. The polymer(s) can also act to prevent the recrystallization of the drug, maintain drug supersaturation, and provide improved physical stability of API in accelerated temperature and humidity conditions which can increase the overall shelf-life of the drug product. The ADS can include the composition including one or more active agents and a stabilizing agent. In some embodiments, the dispersion can include a solubilizing agent. For example, the ADS can include 1-25% w/w active agents, 25-95% w/w stabilizing agent, and 0-10% w/w solubilizing agent. For example, the ADS can include 1, 5, 10, 15, 20, 25% w/w or more active agent(s). For example, the ADS can include 25, 30, 35, 40, 45, 50, 55, 60, 65, 70, 75, 80, 85, 90, 95% w/w or more stabilizing agent(s). For example, the ads can include 0.5, 1, 2, 3, 4, 5, 6, 7, 8, 9, 10% w/w or more solubilizing agent(s).

In some embodiments, the formulation can be in the form of a tablet including the SDD. In some embodiments, the tablet can include one or more fillers, disintegrants, lubricants, and/or glidants. For example, the tablet can include 25-95% w/w spray dried dispersion, 1-75% w/w filler, 0-10% w/w disintegrant, 0-10% lubricant, and/or 0-10% glidant. For example, the table can include 125, 30, 35, 40, 45, 50, 60, 65, 70, 75, 80, 85, 90, 95% w/w or more SDD. For example, the tablet can include 1, 10, 20, 30, 40, 50, 60 70, 75% w/w or more filler. For example, the tablet can include 0.5, 1, 2, 3, 4, 5, 6, 7, 8, 9, 10% w/w or more disintegrant(s). For example, the tablet can include 0.5, 1, 2, 3, 4, 5, 6, 7, 8, 9, 10% w/w or more lubricant(s). For example, the tablet can include 0.5, 1, 2, 3, 4, 5, 6, 7, 8, 9, 10% w/w or more glidant(s). In some embodiments, the tablet can be coated so that the formulation is in the form of a coated tablet. For example, the coated tablet can include one or more enteric coating polymers, anti-tacking agents, plasticizers, and or further vehicles.

In a specific embodiment, the formulation can include an SDD with the API, microcyrstalline cellulose, croscarmellose sodium, magnesium stereate, colloidal silicon dioxide, and/or the like. In another specific embodiment, the formulation can include an SDD with the API, Parteck M100 (mannitol), microcyrstalline cellulose, croscarmellose sodium, magnesium stereate, sodium stearyl fumarate, colloidal silicon dioxide, and/or the like.

### Methods of Making the Formulation

Some embodiments of the invention relate to methods of making any of the formulations provided herein.

Some embodiments of the invention relate to methods of making a tablet formulation. The method can include one or more steps of spray drying, secondary drying, blending, roller compaction, milling, lubrication, tableting, and/or enteric coating.

The spray drying step can include mixing one or more active agents with one or more inactive ingredients and spray drying using solvent(s). A spray dryer can be used wherein crystalline API is converted into amorphous stable spray dried dispersion. In some embodiments, the dispersion can be stable for 12 weeks under accelerated conditions (temperature of 40° C. and relative humidity of 75%). In other embodiments, the dispersion can be stable for 11, 10, 9, or 8 weeks.

The secondary drying step can include drying the spray dried material in a vacuum drying oven to evaporate solvent residue and achieve desired moisture level.

The blending step can include further mixing the spray dried API+excipient combination with other inactive ingredient(s) to obtain a blended API+excipient mixture.

The roller compaction step can include roller compacting the blended mixture using a roller compactor.

The milling step can include milling the roller compacted material to achieve uniform particle size.

The lubrication step can include lubricating the milled material in a blender to improve flowability.

The tableting step can include compressing the lubricated blend into a tablet.

The enteric coating step can include coating the tablet to bypass the stomach to achieve targeted drug release in lower GI tract.

### Methods of Treatment

Some embodiments of the invention relate to methods for preventing or alleviating a gastrointestinal disorder. In some embodiments, the method can include administering any of the formulations described herein to a subject.

For treatment of gastrointestinal disorders, the compositions/formulations of the invention are preferably administered orally, e.g., as a tablet or cachet containing a predetermined amount of the active ingredient(s), pellet, gel, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a liposomal formulation (see, e.g., EP 736299), or in some other form.

Orally administered compositions can include binders, lubricants, inert diluents, lubricating, surface active or dispersing agents, flavoring agents, and humectants. Orally administered formulations such as tablets can optionally be coated or scored and can be formulated so as to provide sustained, delayed, or controlled release of the active ingredient therein.

In some embodiments, the gastrointestinal disorder can be selected from but is not limited to inflammatory bowel syndrome (IBS), gastrointestinal motility disorders, functional gastrointestinal disorders, gastroesophageal reflux disease (GERD), duodenogastric reflux, Crohn's disease, ulcerative colitis, inflammatory bowel disease (IBD), functional heartburn, dyspepsia (including functional dyspepsia or nonulcer dyspepsia), gastroparesis, chronic intestinal pseudo-obstruction (or colonic pseudo-obstruction), and/or the like.

In some embodiments, the administering step can be by oral administration. For example, the composition can be delivered orally in the form of a tablet in an amount sufficient to prevent or alleviate the gastrointestinal disorder. In some embodiments, the tablet can be administered to a subject, but not limited to less than 1, 2, 3, 4, 5, or more times a day.

### EXAMPLES

### Example 1

Pharmacokinetic Study of CBD, CBGA and CBDA in Male Sprague Dawley Rats after Intravenous and Oral Administrations with a Composite API using Different Formulations

This example outlines the general approach that was taken to conduct an in vivo study in male Sprague Dawley (SD) rats so that the pharmacokinetics of CBD, CBGA and CBDA were determined.

The purpose of the experiments was to conduct an in vivo study in 5 groups of male Sprague Dawley (SD) rats to obtain the pharmacokinetic parameters of CBD, CBGA, and CBDA using a composite API in different formulations. Specifically:

Group 1 animals (n=3 male rats) were administered a single intravenous (IV) dose of composite API (formulation 1) at 1 mg/kg.

Group 2-5 animals (n=3 male rats per group) were administered a single oral (PO) dose of composite API at 25 mg/kg (API equivalent dose) in different formulations as shown in the table below.

Blood samples were collected at various time points from each animal up to 24 h post-dose and plasma samples were obtained after centrifuging blood.

The concentrations of the compounds in plasma samples were determined using LC-MS/MS analysis (3 in 1 assay). PK parameters of each compound were obtained using Phoenix® WinNonlin® software (version 8.1).

TABLE 1
Study Groups

| | | Dose | Dose | No. of | |
|---|---|---|---|---|---|
| Group | Formulation | Route | (mg/kg) | Males | Time points |
| 1 | Formulation 1: | IV | 1 | 3 | 5, 15 and 30 |
| | Composite API* | | | | min and 1, 2, |
| | | | | | 4, 8 and 24 h |
| 2 | Formulation 2: | PO | 25 | 3 | 15 and 30 min |
| | Composite API | | | | and 1, 2, 4, 8 |
| 3 | Formulation 3: | PO | 25 | 3 | and 24 h |
| | Composite | | | | |
| | API:Kollidon VA64 | | | | |
| | (15:85, w/w) | | | | |
| 4 | Formulation 4: | PO | 25 | 3 | |
| | Composite | | | | |
| | API:Kollidon | | | | |
| | VA64:SLS | | | | |
| | (15:80:5, w/w/w) | | | | |
| 5 | Formulation 5: | PO | 25 | 3 | |
| | Composite | | | | |
| | API:Kollidon | | | | |
| | VA64:TPGS | | | | |
| | (15:80:5, w/w/w) | | | | |

*Composite API = CBD:CBG:CBDA (60:20:20, w/w/w)

### Regulatory and Safety

This study was conducted in accordance with the applicable Frontage Standard Operating Procedures (SOPs) and IACUC protocols. All general laboratory safety procedures apply.

### Materials

### Test Article Information

Composite API formulations were stored at 4° C. until used.

TABLE 2
Compound Information

| Compound | | Lot Number | Parent MW | BEW |
|---|---|---|---|---|
| Composite API | CBD | 2022-0033-012 | 314.5 g/mol | 1.00 |
| [CBD:CBGA:CBDA | CBGA | | 316.5 g/mol | 1.00 |
| (60:20:20, w/w/w)] | CBDA | | 358.5 g/mol | 1.00 |

BEW: Base equivalent weight.
MW: Molecular weight.

TABLE 1
Composite API Formulation Information

| | Formulation 1 (Composition per tube) | | |
|---|---|---|---|
| | 1 mg/kg | | |
| Composite API for IV | | Amount | No. of |
| Lot Number 2022-0033-010A | Ratio | (mg) | Tubes |
| Composite API | 100% | 0.5 | 12 |
| Total solids per tube | | 0.5 | |
| | Formulation 2 (Composition per tube) | | |
| | 25 mg/kg | | |
| Composite API for PO | | Amount | No. of |
| Lot Number 2022-0033-010B | Ratio | (mg) | Tubes |
| Composite API | 100% | 12.0 | 5 |
| Total solids per tube | | 12.0 | |

| | Formulation 3 (Composition per tube) | | |
|---|---|---|---|
| Composite API:Kollidon VA64 | 25 mg/kg API equivalent | | |
| (15:85) for PO | | Amount | No. of |
| Lot Number 2022-0033-010C | Ratio | (mg) | Tubes |
| Composite API | 15% | 12.0 | 20 |
| Kollidon VA64 | 85% | 68.0 | |
| Total solids per tube | | 80.0 | |

| | Formulation 4 (Composition per tube) | | |
|---|---|---|---|
| Composite API:Kollidon | 25 mg/kg API equivalent | | |
| VA64:SLS (15:80:5) for PO | | Amount | No. of |
| Lot Number 2022-0033-010D | Ratio | (mg) | Tubes |
| Composite API | 15% | 12.0 | 20 |
| Kollidon VA64 | 80% | 64.0 | |
| SLS | 5% | 4.0 | |
| Total solids per tube | | 80.0 | |
| | Formulation 5 (Composition per tube) | | |
| Composite API:Kollidon | 25 mg/kg API equivalent | | |
| VA64:TPGS (15:80:5) for PO | | Amount | No. of |
| Lot Number 2022-0033-010E | Ratio | (mg) | Tubes |
| Composite API | 15% | 12.0 | 20 |
| Kollidon VA64 | 80% | 64.0 | |
| TPGS | 5% | 4.0 | |
| Total solids per tube | | 80.0 | |

### Dose Formulation Preparation

The vehicle was DMSO: ethanol: PEG400: saline (10:10:40:40, v/v/v/v) for IV administration and 50 mM phosphate buffer, pH 6.8 with 0.1% Tween®80 for PO administrations.

### In-Life Study

The in-life pharmacokinetic study in rats was conducted at Frontage Lab, 700 Pennsylvania Drive, Exton, PA 19341. Male Sprague Dawley rats weighing between 250-350 grams, were obtained from Charles River. Group 1 animals were cannulated with jugular vein and femoral artery catheter for IV dose and blood collection. Group 2-5 animals were cannulated with femoral artery catheter for blood collection. Blood/plasma samples were collected via Culex® Automated Blood Sampling System. The details of the study are given below.

TABLE 4
Animal Information

| | |
|---|---|
| Supplier | Charles River Laboratories |
| Species (Strain) | SD (Sprague Dawley) |
| Sex | Male |
| Approx. weight & age range | 250-350 g |
| at dosing | |
| Number to be dosed | 3 rats per group, 15 rats in total |
| Method of Identification | Cage Card, Tag # on tail |
| Availability of Food/Water | IV: Food and water ad libitum |
| | PO: Fasted overnight with food available |
| | after the 4 h sample collection/water ad |
| | libitum |
| Housing | Polycarbonate solid bottom rat cages |

TABLE 5
Dosing and Sample Collection

| | |
|---|---|
| Dose: | Group 1: 1 mg/kg formulation 1 |
| | Group 2: 25 mg/kg formulation 2 |
| | Group 3: 166.7 mg/kg formulation 3 |
| | (25 mg/kg API equivalent) |
| | Group 4: 166.7 mg/kg formulation 4 |
| | (25 mg/kg API equivalent) |
| | Group 5: 166.7 mg/kg formulation 5 |
| | (25 mg/kg API equivalent) |
| Dose concentration | Group 1: 1 mg/mL |
| | Group 2: 2.5 mg/mL |
| | Groups 3-5: 16.67 mg/mL |
| Vehicle | IV: |
| | PO: 50 mM phosphate buffer, pH 6.8 |
| | with 0.1% Tween ®80 |
| Route of administration | Group 1: IV bolus injection |
| | Groups 2-5: oral gavage |
| Dose volume (mL/kg): | IV: 1 mL/kg |
| | PO: 10 mL/kg |
| Frequency of dosing: | Single |
| Volume of blood collected | Serial bleed: 250 μL per time points |
| Time points of | Group 1: 5, 15, 30, 60, 120, 240, 480 and |
| blood/plasma*: Samples | 1440 min |
| | Groups 2-5: 15, 30, 60, 120, 240, 480 and |
| through femoral artery | 1440 min |
| Anticoagulant | K₂-EDTA |

*Plasma samples will be prepared within 30 min of collecting blood.

### Sample Collection and Processing

Blood samples up to 24 h were collected automatically via Culex® Automated Blood Sampling System in tubes containing potassium EDTA anticoagulant. Plasma was harvested by centrifugation within 30 min of collection. Aliquots of dosing solutions (kept frozen at −20° C.) were analyzed at the same time with plasma samples by LC-MS/MS.

### Method Development

An LC-MS/MS method based on multiple reaction monitoring (MRM) of fragment ions was developed to perform quantitative analysis of CBD, CBGA and CBDA in rat plasma (3 in 1 assay). A target LLOQ of 1 ng/mL is desired for higher sensitivity.

### Analysis of Samples

Aliquots of dose formulation were diluted appropriately and analyzed by LC-MS/MS to obtain the concentrations of the analyte in the dosing solutions.

The plasma samples were thawed at room temperature before analyzing. Acetonitrile (or another organic solvent) containing an internal standard were added to plasma to precipitate proteins. The supernatant was either dried and reconstituted with the HPLC mobile phase or further diluted with water prior to analysis by LC-MS/MS. The assay consisted of two separate standard curves, one placed at the beginning of the analytical run and the other arranged towards the end of the sample analysis. Three levels of QCs (low, medium and high) were used to ensure reliability of the assay. The concentrations of the analyte present in plasma was determined using the standard calibration curves prepared with control rat plasma.

### Pharmacokinetics Report

The measured plasma concentrations of CBD, CBGA and CBDA were used to obtain the PK parameters using Phoenix® WinNonlin® software (version 8.1).

### Results

IV dose was injected directly into the systemic circulation. The bioavailability of an IV dose is by definition 100 percent (F=1). Bioavailability is expressed as the percentage of the total drug dose administered that reaches the circulation and becomes available in the body. Formula 1 was administered intravenously and used as a control group. Formulas 2, 3, 4 and 5 were administered by oral route. Formula 2, composite API, showed the highest bioavailability compared to formulas 3, 4 and 5 when administered orally. All formulations tested showed increased half-life with PO compared to IV administration.

TABLE 6

| | Formula | Formula | Formula | Formula | Formula |
|---|---|---|---|---|---|
| | 1 (IV) | 2 (PO) | 3 (PO) | 4 (PO) | 5 (PO) |
| CBD | | | | | |
| Elimination | 0.79 | 3.35 | 2.81 | 1.89 | 2.17 |
| half-life | | | | | |
| (in hours) | | | | | |
| Mean Tmax | | 0.33 | 0.42 | 0.33 | 0.33 |
| (hr) | | | | | |
| Cmax (ng/mL) | | 120 | 91.1 | 103 | 96.2 |
| AUCInf | | 243 | 127 | 149 | 123 |
| (h*ng/mL) | | | | | |
| F (%)* | | 22 | 11 | 14 | 11 |
| CBGA | | | | | |
| Elimination | 4.88 | 5.59 | 4.63 | 4.35 | 4.54 |
| half-life | | | | | |
| (in hours) | | | | | |
| Mean Tmax | | 1.67 | 1.67 | 1 | 0.5 |
| (hr) | | | | | |
| Cmax (ng/mL) | | 6010 | 2560 | 2150 | 3230 |
| AUCInf | | 42100 | 14500 | 12800 | 17400 |
| (h*ng/mL) | | | | | |
| F (%)* | | 22 | 9 | 7 | 10 |
| CBDA | | | | | |
| Elimination | 0.34 | 3.47 | 3.02 | 3.34 | 2.54 |
| half-life | | | | | |
| (in hours) | | | | | |
| Mean Tmax | | 0.19 | 0.33 | 0.33 | 0.25 |
| (hr) | | | | | |
| Cmax (ng/mL) | | 741 | 492 | 619 | 639 |
| AUCInf | | 1320 | 660 | 874 | 704 |
| (h*ng/mL) | | | | | |
| F (%)* | | 84 | 38 | 51 | 43 |

Bioavailability (F) was estimated using AUClast and the mean AUClast (41.5 h*ng/mL) following a mean IV bolus dose of 0.60 mg/kg.
Formula 1 (IV): Formulation 1 (Composite API)
Formula 2 (PO): Formulation 2 (Composite API)
Formula 3 (PO): Formulation 3 (Composite API: Kollidon VA64 (15:85, w/w)
Formula 4 (PO): Formulation 4 (Composite API: Kollidon VA64: SLS (15:80:5, w/w/w)
Formula 5 (PO): Formulation 5 (Composite API: Kollidon VA64: TPGS (15:80:5, w/w/w)

### Example 2

### Example Formulations

This Example provides example formulations of the invention.

TABLE 7

| | | | | |
|---|---|---|---|---|
| Ingredient | mg | % w/w | Function | Range |

Spray dried dispersion (API + Polyer mix to stabilize crystalline API in the amorphous form)

| | | | | |
|---|---|---|---|---|
| CBD (Isolate) | 15 | 9 | Active | N/A |
| CBG (Isolate) | 5 | 3 | Active | N/A |
| CBDA (Isolate) | 5 | 3 | Active | N/A |
| Kollidon VA 64 | 137.5 | 82.5 | Polymer | 77.5 to 88.5 |
| (Copovidone) | | | (stabilizing agent) | |
| Sodium Lauryl | 4.17 | 2.5 | Plasticizer, | 2 to 3 |
| Sulfate | | | solublilizing agent | |
| Total | | 100 | | |

Formulation 1

| | | | | |
|---|---|---|---|---|
| API SDD | 166.67 | 69.44 | | 10 to 80 |
| Microcrystalline | 57.74 | 24.1 | Filler | 10 to 50 |
| Cellulose OR | | | | |
| Lactose | | | | |
| Croscarmellose | 12 | 5 | Disintegrant | 2 to 10 |
| sodium OR sodium | | | | |
| starch glycolate | | | | |
| Magnesium Stearate | 2.4 | 1 | Lubricant | 0.5 to 2.0 |
| Colloidal Silicon | 1.2 | 0.5 | Glidant | 0.2 to 2.0 |
| Dioxide | | | | |
| Total Core Tablet | 240.01 | 100.04 | | |
| Weight (mg) | | | | |

Formulation 2

| | | | | |
|---|---|---|---|---|
| API SDD | 166.7 | 26.5 | | N/A |
| Mannitol Or | 166.7 | 26.5 | Filler | 10 to 50 |
| Lactose Or MCC | | | | |
| Microcrystalline | 226 | 35.9 | Filler | 10 to 50 |
| Cellulose PH 101 | | | | |
| OR Lactose Or | | | | |
| Mannitol | | | | |
| Croscarmellose | 35 | 5.6 | Disintegrant | 3 to 10 |
| sodium OR sodium | | | | |
| starch glycolate | | | | |
| Magnesium Stearate | 3.2 | 0.5 | Lubricant | 0.5 to 2.0 |
| Sodium Stearyl | 30 | 4.8 | Lubricant | 1 to 5% |
| Fumarate | | | | |
| Colloidal Silicon | 2.5 | 0.4 | Glidant | 0.2 to 2.0 |
| Dioxide | | | | |
| Total Core Tablet | 630.1 | 100.2 | | |
| Weight (mg) | | | | |

Final Finished Product (Coated Tablet)

| | | | | |
|---|---|---|---|---|
| Core Tablet | 240 | 90.9 | N/A | 80 to 93% |
| Eudragit L30D 55 | 14.4 | 5.45 | Enteric coating | 3.5 to 7.3 |
| OR Eudragit FS 30D | | | polymer | |
| OR HPMC Phthalate | | | | |
| HP 55 | | | | |
| Titanium Dioxide | 20.0 | 1.82 | Opacifier/Pigment | 0.8 to 2.7 |
| Talc | 19.4 | 1.76 | Glidant | 0.8 to 2.7 |
| Tri Ethyl citrate/ | 0.6 | 0.05 | Plasticizer | 0.04 to 0.06 |
| Polyethylene glycol | | | | |
| Water (For Eudragit | 176 | Qs (to make 12% | Vehicle (evaporated) | 10 to 15% |
| L30D55 OR FS 30D) | mg/tab | w/w of solution) | | |
| Dichloromethane:Ethanol | 225.6 | Qs (to make 6% | Vehicle (evaporated) | 5 to 7% |
| (50:50) (For HPMC | mg/tab | w/w of solution) | | |
| pthalate HP55) | | | | |

### Example 3

Experiments were done to test for suitable polymers for an ASD using Hansen Solubility Parameters for CBD, CBGA and CBDA. Hansen Solubility Parameters (HSP) are a set of three parameters used to describe the solubility and compatibility of various substances. The three parameters are dispersion forces (6d), polar forces (6p), and hydrogen bonding forces (6h). Measuring Hansen Solubility Parameters typically involves a combination of experimental and theoretical approaches. The miscibility between an API and polymer was tested based on their solubility parameters. Maximum miscibility limits were estimated based on Gibbs Free Energy Calculations. Polymer matrix choice is driven by maximum miscibility of the API and polymer. The following polymers were tested for this experiment.

TABLE 8

| Polymer | δtpolymer − | δtpolymer − | δtpolymer − |
|---|---|---|---|
| Excipients | δtAPI (CBD) | δtAPI (CBGA) | δtAPI (CBDA) |
| Soluplus | 0.5 | 0.3 | 0.1 |
| Eudragit EPO | 0.8 | 0.6 | 0.2 |
| ethyl cellulose | 0.9 | 0.7 | 0.2 |
| Poloxamer 407 | 1.2 | 1 | 0.4 |
| PVP K30 | 1.4 | 1.2 | 0.4 |
| Poloxamer 188 | 1.4 | 1.2 | 0.5 |
| Eudragit S100 | 1.5 | 1.3 | 0.5 |
| Kollidon VA64 | 2.2 | 2 | 1.2 |
| Eudragit L100 | 2.2 | 2 | 1.2 |
| Eudragit L100-55 | 2.2 | 2 | 1.2 |
| PEG 1500 | 2.5 | 2.3 | 1.5 |
| PEG 6000 | 2.7 | 2.5 | 1.7 |

Based on the HSP calculations, Soluplus, Eudragit EPO, PVP K30, Kollidon VA 64 and Eudragit S100 polymers showed the least solubility parameter differences (most likely to be compatible with API) for the 3 APIs. The lower the Δδt, better the miscibility between API and polymer.

TABLE 9

| | δtpolymer − δtAPI | | |
|---|---|---|---|
| Polymer Excipients | CBD | CBGA | CBDA |
| Soluplus | 0.5 | 0.3 | 0.5 |
| Eudragit EPO | 0.8 | 0.6 | 0.2 |
| Eudragit S100 | 1.5 | 1.3 | 0.5 |
| PVP K30 | 1.4 | 1.2 | 0.4 |
| Kollidon VA 64 | 2.2 | 2 | 1.2 |

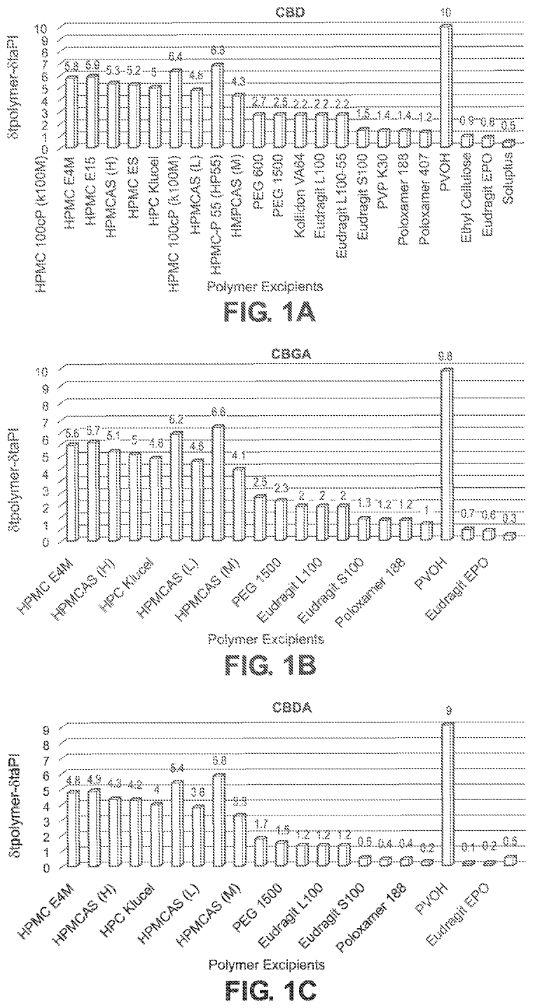

Results are depicted in FIG. 1. In summary, all polymers that were tested are suitable for use in the invention. As known in the field, the tower the Δδt, better the miscibility between API and polymer. However, there are other factors, such as formulation, route of administration, other chemical interactions can influence miscibility of the API in polymer mix.

### Example 4

Experiments were done to test for polymers with the best dissolution behavior for CBD, CBGA, CBDA. Micro evaporation screening experiments were done by dissolving API and polymer in an organic solvent and mixed in centrifuge tubes. Then the solvent was removed using vacuum concentrator. Samples were then mixed for 4, 10, and 30 minutes using phosphate buffer pH 6.8 as a reconstitution medium. For Eudragit S100 phosphate buffer pH 7.4 was used as a reconstitution media, citrate buffer pH 4.5 was used for Eudragit EPO. Phosphate buffer pH 6.8 was used for the rest of the polymers. Samples were mixed for 4, 10, and 30 minutes. Analysis was done by UV spectrometry to measure re-dissolution behavior of polymer system.

Several organic solvents were screened for the micro evaporation screening trials including acetone, tetrahydrofuran, dichloromethane, methanol, and ethanol.

Round 1 matrix Screening
30:70 API:Soluplus
30:70 API:Eudragit EPO
30:70 API:Kollidon VA 64
25:75 API:PVP K30
25:75 API:Eudragit S100

Round 2 matrix screening with surfactants

Surfactants/solubilizers (5-10%) were screened for enhancement of spring-parachute effect, which is a strategy used in the pharmaceutical formulation to improve the solubility and bioavailability of poorly-soluble drugs:
PEG 1500
Poloxamer 407 (HLB˜18)
SLS (HLB˜40)
Kolliphor RH40 (HLB−15)
TPGS (HLB˜13)

CBD Miniaturized Screening Experiment—Micro Evaporation Matrices screened:
API:Soluplus (30:70)
API:Eudragit EPO (30:70)
API:PVP K30 (25:75)
API:Eudragit S100 (25:75)
API:Kollidon VA64 (10:90)
API+Kollidon VA64+PEG1500 (10:85:5)
API+Kollidon VA64+SLS (10:85:5)
API+Kollidon VA64+Kolliphor RH40 (10:85:5)
API+Kollidon VA64+TPGS (10:85:5)
API+Kollidon VA64+Poloxamer 407 (10:85:5)

(Each tube contained 1 mg of API)

CBGA Miniaturized Screening Experiment—Micro Evaporation

Matrices screened:
API:Soluplus (30:70)
API:Eudragit EPO (30:70)
API:PVP K30 (25:75)
API:Eudragit S100 (25:75)
API:Kollidon VA64 (15:85)
API+Kollidon VA64+PEG1500 (15:80:5)
API+Kollidon VA64+SLS (15:80:5)
API+Kollidon VA64+Kolliphor RH40 (15:80:5)
API+Kollidon VA64+TPGS (15:80:5)
API+Kollidon VA64+Poloxamer 407 (15:80:5)

(Each tube contained 1 mg of API)

CBDA Miniaturized Screening Experiment—Micro Evaporation Matrices screened:
API:Eudragit EPO (30:70)
API:PVP K30 (30:70)
API:Soluplus (30:70)
API:Eudragit S100 (30:70)
API:Kollidon VA64 (25:75)
API+Kollidon VA64+PEG1500 (25:70:5)
API+Kollidon VA64+SLS (25:70:5)
API+Kollidon VA64+Kolliphor RH40 (25:70:5)
API+Kollidon VA64+TPGS (25:70:5)
API+Kollidon VA64+Poloxamer 407 (25:70:5)

(Each tube contained 1 mg of API).

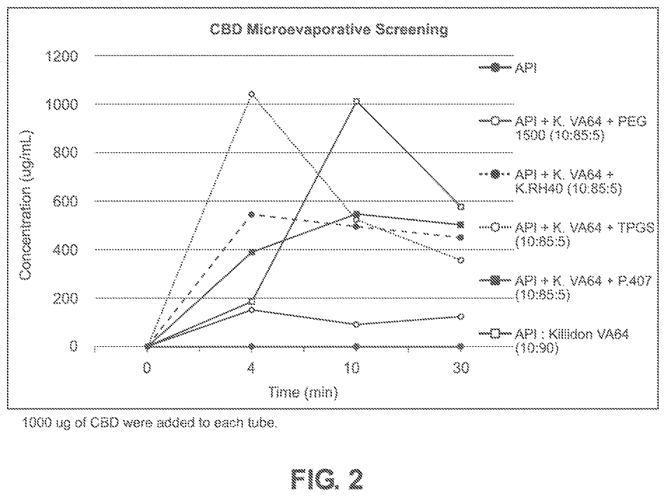

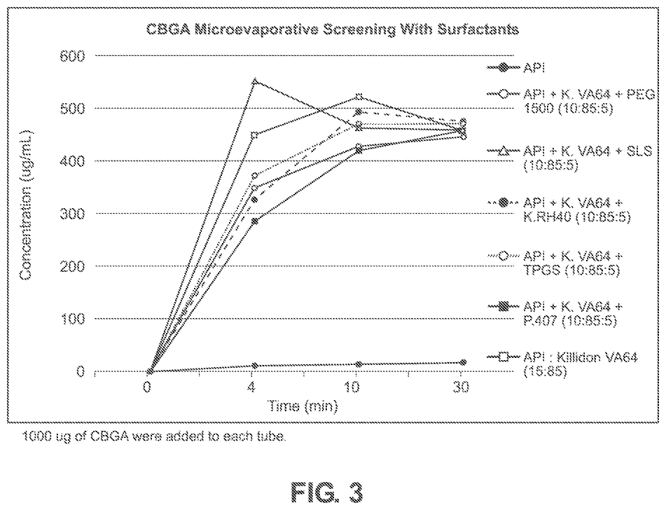

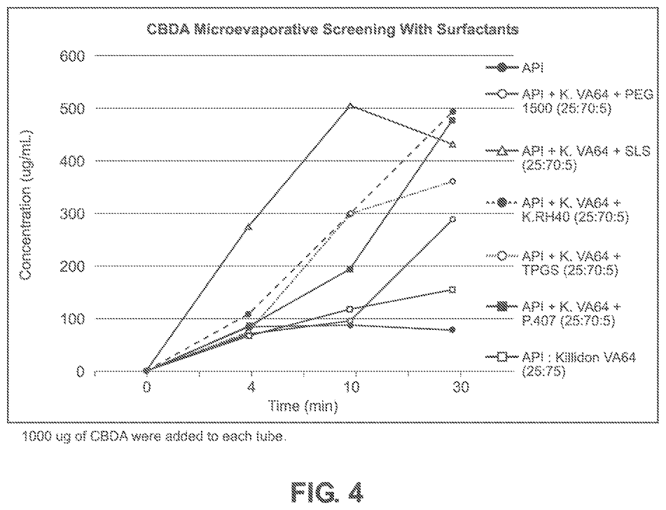

Experiments showed that Kollidon VA64 had the best dissolution behavior of all the polymers under these experimental conditions. For CBD, Kollidon VA64 showed a complete dissolution of the amount of API added at 10 min; Kollidon VA64 with TPGS added shows similar results to polymer alone. Note: SLS results not shown as there was interference from the SLS for absorbance reading. For CBGA, Matrix with Kollidon VA64 showed the best dissolution behavior with the addition of SLS and Poloxamer 407 with a dissolution concentration of 520-550 ug/ml. For CBDA, Matrix with Kollidon VA64 showed the best dissolution behavior with the addition of SLS, Kolliphor RH40 with a dissolution concentration of 490-500 ug/ml at 10 and 30 min timepoints. Note: Additional centrifuging time for matrix with surfactants could have affected results. Results are depicted in FIGS. 2-4. In summary, all polymers that were tested are suitable for use in the invention.

### Example 5

Experiments were done to test non-sink dissolution of various prototypes produced by amorphous solid dispersion method. Non-sink dissolution is a dissolution test performed where the drug concentration in the medium increases as the drug dissolves, and the dissolution rate is affected by the amount of drug that has already dissolved. Non sink dissolution of CBD, CBGA, and CBDA, were done by HPLC. SDD1, SDD2, and SDD3 are replicates of the same formulation.

TABLE 10
SDD composition

| | % w/w | % w/w | Theoretical Batch |
|---|---|---|---|
| Material name | Solids | Solution | Quantity (g) |
| CBD | 9.00 | 0.90 | 7.20 |
| CBGA | 3.00 | 0.30 | 2.40 |
| CBDA | 3.00 | 0.30 | 2.40 |
| Plasdone S-630 | 82.50 | 8.25 | 66.00 |
| Sodium Lauryl | 2.50 | 0.25 | 2.00 |
| Sulfate | | | |
| Methanol | Evaporated | 18.00 | 144.00 |
| Acetone | Evaporated | 72.00 | 576.00 |
| Total Solid Content | 100.00 | 10.00 | 80.00 |
| in Solution | | | |
| Total Batch size of | N/A | 100.00 | 800.00 |
| Solution | | | |

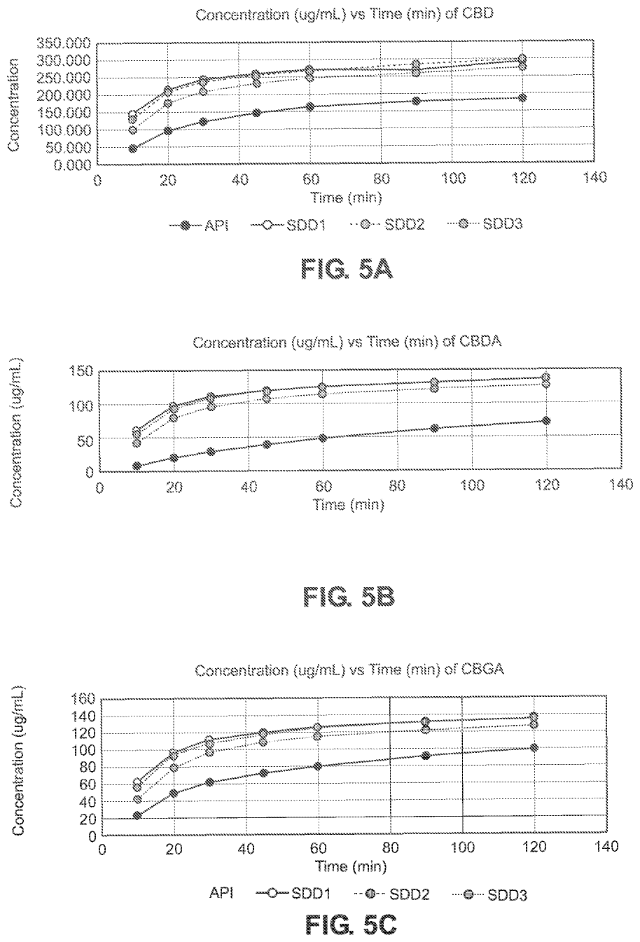

Results are shown in FIG. 5.

TABLE 11

| | | | | | |
|---|---|---|---|---|---|
| Time (min) | API | SDDl | SDD2 | SDD3 | Average |

CBD: Amount (ug/mL)

| | | | | | |
|---|---|---|---|---|---|
| 10 | 45.868 | 145.099 | 129.903 | 98.420 | 124.47 |
| 20 | 94.947 | 214.522 | 207.326 | 176.663 | 199.50 |
| 30 | 121.882 | 242.262 | 236.632 | 209.743 | 229.55 |
| 45 | 146.211 | 258.578 | 253.936 | 230.952 | 247.82 |
| 60 | 162.522 | 270.026 | 267.386 | 247.232 | 261.55 |
| 90 | 177.782 | 267.192 | 282.958 | 260.432 | 270.19 |
| 120 | 184.122 | 294.348 | 296.232 | 275.366 | 288.65 |

CBDA: Amount (ug/mL)

| | | | | | |
|---|---|---|---|---|---|
| 10 | 8.39 | 62.684 | 56.576 | 42.891 | 54.05 |
| 20 | 20.112 | 97.701 | 95.058 | 80.878 | 91.21 |
| 30 | 28.96 | 113.014 | 110.034 | 97.803 | 106.95 |
| 45 | 39.729 | 121.66 | 119.959 | 109.481 | 117.03 |
| 60 | 49.276 | 127.355 | 126.958 | 116.659 | 123.66 |
| 90 | 62.937 | 133.681 | 133.849 | 123.788 | 130.44 |
| 120 | 72.946 | 139.568 | 139.568 | 129.342 | 136.16 |

CBGA: Amount (ug/ml)

| | | | | | |
|---|---|---|---|---|---|
| 10 | 23.365 | 61.744 | 55.942 | 42.584 | 53.42 |
| 20 | 49.054 | 96.195 | 93.631 | 79.55 | 89.79 |
| 30 | 61.557 | 111.085 | 108.022 | 96.673 | 105.26 |
| 45 | 71.91 | 119.498 | 118.048 | 107.995 | 115.18 |
| 60 | 79.837 | 124.975 | 124.819 | 114.794 | 121.53 |
| 90 | 91.055 | 131.345 | 131.263 | 121.607 | 128.07 |
| 120 | 99.329 | 134.798 | 136.537 | 126.754 | 132.70 |

Non-sink dissolutions data showed that spray dried dispersion of API and polymer mix had higher solubility and therefore dissolution rate compared to crystalline API.

### Example 6

Experiments were done to test stability of SDD formulations with CBD, CBDA or CBGA. Table 12 shows up to 12 weeks stability data of spray dried dispersion formulation containing CBD, CBGA, CBDA, Plasdone S-630 and SLS. Methanol and acetone were used as solvents. Buchi min spray-dryer model #B-290 was used to manufacture the product using the following spray drying parameters:

TABLE 12

| Spray Drying Parameter | Target | Actual |
|---|---|---|
| Inlet Temperature (° C.) | 80 ± 20 | 80 ± 20 |
| Outlet Temperature (° C.) | 42-58^{[2]} | 49-53 |
| Aspirator (%) | 90-100 | 100 |
| Gas flow height (mm) | 40-50 | 40 |
| Solution Feed Rate (g/min) ^{[} | 7-10 | ~8 |

Assay/Impurities testing was performed by HPLC. Non-sink dissolution was performed by UV. Water content was measured as per the USP 921 using Karl Fischer. PXRD was performed by X-ray diffraction. Results showed that the product was stable under room temperature as well as accelerated conditions for 12 weeks. No major changes were observed in the product appearance. Water content was less than 3% after 12 weeks. Assay values remained consistent, non-sink dissolution behavior remained mostly unchanged, and all the APIs remained in amorphous form.

Results are provided in the following tables:

TABLE 13
ANALYTICAL 25° C./60% RH

| Test/Attribute | | | | | | | | | | | |
|---|---|---|---|---|---|---|---|---|---|---|---|
| Result Criteria | | | | | | | | | | | |
| T = 0 | | | T = 4 Weeks | | | T = 8 Weeks | | | T = 12 Weeks | | |
| Appearance | | | | | | | | | | | |
| Report Results | | | | | | | | | | | |
| Off-white Powder | | | | | | | | | | | |
| Assay/Impurities | | | | | | | | | | | |
| Report Results | | | | | | | | | | | |
| % | % | % | % | % | % | % | % | % | % | % | % |
| CBD | CBGA | CBDA | CBD | CBGA | CBDA | CBD | CBGA | CBDA | CBD | CBGA | CBDA |
| 92.9 | 99.8 | 102.2 | 91.4 | 101.3 | 103.3 | 96 | 103.1 | 106.1 | 97.2 | 99.2 | 103.4 |

| Non-Sink | | | | | | | | | |
|---|---|---|---|---|---|---|---|---|---|
| Disso- | | | | | | | | | |
| lution - | Report | Time | CBD | Time | CBD | Time | CBD | Time | CBD |
| CBD | Results | (min) | (ug/mL) | (min) | (ug/mL) | (min) | (ug/mL) | (min) | (ug/mL) |
| (n = 3) | | 10 | 124.5 | 10 | 86.9 | 10 | — | 10 | 104 |
| | | 20 | 199.5 | 20 | 137.1 | 20 | — | 20 | 143 |
| | | 30 | 299.6 | 30 | 163 | 30 | — | 30 | 179 |
| | | 45 | 247.8 | 45 | 185.4 | 45 | — | 45 | 208 |
| | | 60 | 261.6 | 60 | 190.8 | 60 | — | 60 | 228 |
| | | 90 | 270.2 | 90 | 203.2 | 90 | — | 90 | 249 |
| | | 120 | 288.7 | 120 | 214.5 | 120 | — | 120 | 269 |
| Non-Sink | | | | | | | | | |
| Disso- | | | | | | | | | |
| lution - | Report | Time | CBDA | Time | CBDA | Time | CBDA | Time | CBDA |
| CBDA | Results | (min) | (ug/mL) | (min) | (ug/mL | (min) | (ug/mL | (min) | (ug/mL |
| (n = 3) | | 10 | 54.1 | 10 | 41.2 | 10 | — | 10 | 42 |
| | | 20 | 91.21 | 20 | 67.7 | 20 | — | 20 | 67 |
| | | 30 | 107 | 30 | 77 | 30 | — | 30 | 85 |
| | | 45 | 117 | 45 | 89 | 45 | — | 45 | 99 |
| | | 60 | 123.7 | 60 | 92.9 | 60 | — | 60 | 107 |
| | | 90 | 130.4 | 90 | 97.6 | 90 | — | 90 | 115 |
| | | 120 | 136.2 | 120 | 103.3 | 120 | — | 120 | 123 |
| Non-Sink | | | | | | | | | |
| Disso- | | | | | | | | | |
| lution - | Report | Time | CBGA | Time | CBGA | Time | CBGA | Time | CBGA |
| CBGA | Results | (min) | (ug/mL) | (min) | (ug/mL | (min) | (ug/mL) | (min) | (ug/mL) |
| (n = 3) | | 10 | 53.4 | 10 | 44 | 10 | — | 10 | 49 |
| | | 20 | 89.8 | 20 | 68.3 | 20 | — | 20 | 68 |
| | | 30 | 105.3 | 30 | 81.8 | 30 | — | 30 | 85 |
| | | 45 | 115.2 | 45 | 93 | 45 | — | 45 | 98 |
| | | 60 | 121.5 | 60 | 97 | 60 | — | 60 | 107 |
| | | 90 | 128.1 | 90 | 102.5 | 90 | — | 90 | 115 |
| | | 120 | 132.7 | 120 | 107.6 | 120 | — | 120 | 124 |
| Water | Report | N.D. | | 2.38% | | 1.84% | | 2.76% | |
| Content | Results | | | | | | | | |
| PXRD | Report | Amorphous | | Amorphous | | Amorphous | | Amorphous | |
| | Results | | | | | | | | |

TABLE 14
ANALYTICAL 40° C./75% RH

| Test/Attribute | | | | | | | | | | | | | | |
|---|---|---|---|---|---|---|---|---|---|---|---|---|---|---|
| Result Criteria | | | | | | | | | | | | | | |
| T = 0 | | | T = 1 Week | | | T = 4 Weeks | | | T = 8 Weeks | | | T = 12 Weeks | | |
| Appearance | | | | | | | | | | | | | | |
| Report Results | | | | | | | | | | | | | | |
| Off-white Powder | | | | | | | | | | | | | | |
| Assay/Impurities | | | | | | | | | | | | | | |
| Report Results | | | | | | | | | | | | | | |
| CBD | CBGA | CBDA | CBD | CBGA | CBDA | CBD | CBGA | CBDA | CBD | CBGA | CBDA | CBD | CBGA | CBDA |
| 92.9 | 99.8 | 102.2 | 84.5 | 99.8 | 101 | 90.6 | 100.3 | 102.4 | 94.4 | 101.3 | 102.9 | 100.4 | 101.5 | 104 |

| Non- | | | | | | | | | | | |
|---|---|---|---|---|---|---|---|---|---|---|---|
| Sink | | | | | | | | | | | |
| Disso- | | | | | | | | | | | |
| lution - | Report | Time | CBD | Time | CBD | Time | CBD | Time | CBD | Time | CBD |
| CBD | Results | (min) | (ug/mL) | (min) | (ug/mL) | (min) | (ug/mL) | (min) | (ug/mL) | (min) | (ug/mL) |
| (n = 3) | | 10 | 124.5 | 10 | 132.3 | 10 | 86 | 10 | | 10 | 100 |
| | | 20 | 199.5 | 20 | 185.7 | 20 | 139.7 | 20 | | 20 | 152 |
| | | 30 | 299.6 | 30 | 213.7 | 30 | 166.8 | 30 | | 30 | 192 |
| | | 45 | 247.8 | 45 | 238 | 45 | 191.2 | 45 | | 45 | 220 |
| | | 60 | 261.6 | 60 | 252 | 60 | 204.3 | 60 | | 60 | 242 |
| | | 90 | 270.2 | 90 | 262 | 90 | 216.4 | 90 | | 90 | 263 |
| | | 120 | 288.7 | 120 | 282.3 | 120 | 230.3 | 120 | | 120 | 277 |
| Non- | | | | | | | | | | | |
| Sink | | | | | | | | | | | |
| Disso- | | | | | | | | | | | |
| lution- | Report | Time | CBDA | Time | CBDA | Time | CBDA | Time | CBDA | Time | CBDA |
| CBDA | Results | (min) | (ug/mL) | (min) | (ug/mL) | (min) | (ug/mL) | (min) | (ug/mL) | (min) | (ug/mL) |
| (n = 3) | | 10 | 54.1 | 10 | 57.7 | 10 | 43.5 | 10 | | 10 | 43 |
| | | 20 | 91.21 | 20 | 82 | 20 | 68.2 | 20 | | 20 | 69 |
| | | 30 | 107 | 30 | 95.3 | 30 | 80.6 | 30 | | 30 | 88 |
| | | 45 | 117 | 45 | 103.3 | 45 | 91.9 | 45 | | 45 | 101 |
| | | 60 | 123.7 | 60 | 110 | 60 | 97.4 | 60 | | 60 | 110 |
| | | 90 | 130.4 | 90 | 115.5 | 90 | 102.1 | 90 | | 90 | 118 |
| | | 120 | 136.2 | 120 | 118.3 | 120 | 108 | 120 | — | 120 | 124 |
| Non- | | | | | | | | | | | |
| Sink | | | | | | | | | | | |
| Disso- | | | | | | | | | | | |
| lution - | Report | Time | CBGA | Time | CBGA | Time | CBGA | Time | CBGA | Time | CBGA |
| CBGA | Results | (min) | (ug/mL) | (min) | (ug/mL) | (min) | (ug/mL) | (min) | (ug/mL) | (min) | (ug/mL) |
| (n = 3) | | 10 | 53.4 | 10 | 59.3 | 10 | 44.9 | 10 | | 10 | 45 |
| | | 20 | 89.8 | 20 | 84.3 | 20 | 70.2 | 20 | | 20 | 71 |
| | | 30 | 105.3 | 30 | 97.7 | 30 | 83 | 30 | | 30 | 90 |
| | | 45 | 115.2 | 45 | 106 | 45 | 84.2 | 45 | | 45 | 103 |
| | | 60 | 121.5 | 60 | 112.3 | 60 | 99.8 | 60 | | 60 | 112 |
| | | 90 | 128.1 | 90 | 118 | 90 | 104.5 | 90 | | 90 | 120 |
| | | 120 | 132.7 | 120 | 121 | 120 | 109.7 | 120 | | 120 | 126 |

| | | | | | | |
|---|---|---|---|---|---|---|
| Water | Report | N.D. | 2.55% | 2.28% | 2.10% | 2.99% |
| Content | Results | | | | | |
| PXRD | Report | Amorphous | Amorphous | Amorphous | Amorphous | Amorphous |
| | Results | | | | | |

SDD was further compressed into tablets. Two formulations were evaluated (Formulation X and Formulation Y).

For Formulation X, Blending was performed in Turbula blender followed by roller compaction using TFC roller compactor. Tablets were compressed on a single punch manual press.

### Formulation X

TABLE 15

| | | | Theoretical Batch |
|---|---|---|---|
| Material name | mg/tablet | % w/w | Quantity (g) |
| API SDD | 166.67 | 69.4% | 34.72 |
| Microcrystalline cellulose | 57.74 | 24.1% | 12.03 |
| 101 (Avicel PH101) | | | |
| Croscarmellose sodium | 12 | 5.0% | 2.50 |
| (Ac-Di-Sol) | | | |
| Magnesium Stereate | 2.4 | 1.0% | 0.50 |
| Colloidal Silicon Dioxide | 1.2 | 0.5% | 0.25 |
| (Cab-O-Sil M5P) | | | |
| Total | 240.01 | 100.0% | 50.00 |

Physical testing of the final blend was performed, and results are reported below.

TABLE 16

| | |
|---|---|
| Blend Volume (mL) | 45.0 |
| Blend Weight (g) | 21.0 |
| Volume 10 taps (mL): | 41 |
| Volume 500 taps: | 32 |
| Volume 1250 taps: | 31 |
| Bulk density (g/ml) | 0.467 |
| Tapped density | 0.677 |
| Hausner's ratio | 1.452 |
| Carr's index | 31.111 |

Particle size of the final blend was measured using Malvern 3000 and results are shown below:

TABLE 17

| PSD | |
|---|---|
| D50 | 128 um |
| D90 | 1260 um |

In process testing of the core tablets was done and results are shown below:

TABLE 18
Manual compression, single punch press
Tooling size 0.375

| Compression | | | | Initial | Final | | |
|---|---|---|---|---|---|---|---|
| force | Weight | Thickness | Hardness | weight | weight | Friability | DT |
| (lbs) | (mg) | (mm) | (kp) | (g) | (g) | (%) | (min) |
| 1000 | 240 | 3.99 | 10 | 1187 | 1188 | −0.08% | >15 min |
| | 239 | 4.11 | 8.1 | | | | |
| | 240 | 4.01 | 8.7 | | | | |
| | 239 | 4.05 | 8.8 | | | | |
| | 240 | 4 | 8.9 | | | | |
| 2000 | 239 | 3.5 | 17.7 | 1187 | 1187 | 0.00% | >15 min |
| | 240 | 3.7 | 16.1 | | | | |
| | 240 | 3.8 | 18.9 | | | | |
| | 239 | 3.33 | 19.8 | | | | |
| | 239 | 3.78 | 18.8 | | | | |
| 1500 | 239 | 3.77 | 13.1 | 1185 | 1186 | −0.08% | >15 min |
| | 239 | 3.83 | 15.3 | | | | |
| | 240 | 3.77 | 15.3 | | | | |
| | 240 | 3.8 | 15.1 | | | | |
| | 239 | 3.78 | 15.1 | | | | |

| | | |
|---|---|---|
| 500-600 | 4-5 | >13 min |

### Formulation Y

For Formulation y, Blending was performed in Turbula blender followed by roller compaction using TFC roller compactor. Tablets were compressed on a rotary tablet press (Korsch XL 100).

TABLE 19

| | | | Theoretical Batch |
|---|---|---|---|
| Material name | mg/tablet | % w/w | Quantity (g) |
| API SDD | 166.7 | 26.5% | 13.23 |
| Parteck M100 | 166.7 | 26.5% | 13.23 |
| Microcrystalline cellulose | 226.0 | 35.9% | 17.94 |
| 101 (Avicel PH101) | | | |
| Croscarmellose sodium | 35.0 | 5.6% | 2.78 |
| (Ac-Di-Sol) | | | |
| Magnesium Stereate | 3.2 | 0.5% | 0.25 |
| Sodium Stearyl Fumarate | 30.0 | 4.8% | 2.38 |
| Colloidal Silicon Dioxide | 2.5 | 0.4% | 0.20 |
| (Cab-O-Sil M5P) | | | |
| Total | 630.0 | 100.0% | 50.00 |

Physical testing of the final blend was performed, and results are reported below.

TABLE 20

| | |
|---|---|
| Blend Volume (mL) | 12.0 |
| Blend Weight (g) | 8.4 |
| Volume 10 taps (mL): | |
| Volume 500 taps: | 10 |
| Volume 1250 taps: | 10 |
| Bulk density (g/ml) | 0.700 |
| Tapped density | 0.840 |
| Hausner's ratio | 1.200 |
| Carr's index | 16.667 |

In process testing of the core tablets was performed and results are shown below.

TABLE 21
Korsch XL100
Tooling size 0.4375

| Compression | | | | Initial | Final | | |
|---|---|---|---|---|---|---|---|
| force | Weight | Thickness | Hardness | weight | weight | Friability | DT |
| (lbs) | (mg) | (mm) | (kp) | (g) | (g) | (%) | (min) |
| 500 | 0.63 | 7.47 | 3.4 | 3128 | 3102 | 0.83% | 10-12 |
| | 0.629 | 7.53 | 3.2 | | | | |
| | 0.632 | 7.45 | 3.9 | | | | |
| | 0.631 | 7.5 | 3.2 | | | | |
| | 0.63 | 7.6 | 3.7 | | | | |
| 800 | 0.629 | 7.3 | 6.5 | 3142 | 3122 | 0.64% | 10 |
| | 0.629 | 7.22 | 7.9 | | | | |
| | 0.63 | 7.18 | 6.9 | | | | |
| | 0.63 | 7.01 | 7.5 | | | | |
| | 0.63 | 7.15 | 6.5 | | | | |
| 1100 | 0.629 | 6.82 | 10.6 | 3142.5 | 3137.5 | 0.16% | 12-15 |
| | 0.63 | 6.55 | 12.2 | | | | |
| | 0.63 | 6.72 | 11.6 | | | | |
| | 0.63 | 6.7 | 11.7 | | | | |
| | 0.63 | 6.82 | 11.8 | | | | |

There were no major issues reported for either formulation. Formulation Y demonstrated better flow properties compared to Formulation X, as indicated by, for example, the Carr's index value. Both formulations met the friability test criteria and disintegration time was comparable.

### Example 7

Patients are treated using formulations and methods of the invention. All patients have a confirmed diagnosis of IBS by their 01 doctor. Patients included in the sample have data collected from hospital medical records, outpatient exams, and long-term regular follow ups. Sample size=225 patients. After a 12-week treatment plan, a large majority of patients in the cohort report improvement in over more of these symptom: improvement in abdominal bloating and distention, improvement in abdominal cramping, improvement in nausea, and improvement in diarrhea.

### Example 8

This Example provides details of an example method of producing an embodiment of the invention:

TABLE 22

| Process | | Critical process | | |
|---|---|---|---|---|
| step | Equipment | parameters | Range | |
| Spray | Spray dryer | Spray rate | Different based on the equipment | |
| drying | (Buchi 290, MS | Feed solution | 30 min to 4 hr | |
| | 35, MS 150) | Mixing time | (until it become clear) | |
| | | Outlet temperature | 40 to 55 | C. |
| Secondary | Oven | Temperature | 35 to 40 | C. |
| drying | | | | |
| Blending | Blender | Blending time | 5 to 15 | minutes |
| Roller | Roller compactor | Compaction force | 3 to 10 | kN/cm |
| compaction | (Gerties, Vector) | Roll Gap | 1 to 3 | mm |
| | | Roll speed | 2 to 4 | rpm |
| Milling | | Screen size | 0.8 to 1.5 | mm |
| Lubrication | Blender | Lubrication time | 2 to 8 | minutes |
| Tableting | Compression | Compression force | 10 to 30 | kN |
| | press | Turret speed | 20 to 40 | rpm |
| | (Korsch X100) | | | |

| | | | |
|---|---|---|---|
| Enteric | Coater | Product | 35 to 50 (Depends on polymer) |
| coating | (Vector/Ohara) | temperature | |
| | | Exhaust | 35 to 50 (Depends on polymer) |
| | | temperature | |
| | | Spray rate | Varies based on Pan size |

### Process Detail:

1. Dissolve CBD, CBDA, CBGA in Methanol:Acetone mixture to form clear solution.
2. Add Copovidone in to step #1 and mix to dissolve it completely.
3. Spray the step #2 feed solution in spray drier at desired process parameters mentioned in above table.
4. Collect spray dried powder and dry in to Oven
5. Mix the SDD with Filler, Disintegrant and mix in blender for appropriate time.
6. Perform roller compaction of step #5 blend.
7. Perform Lubrication of step #6 using blender.
8. Compress the step #7 blend using compression press.
9. Disperse Enteric polymer, Talc, Tio2 and Plasticizer in the vehicle mentioned in above table.
10. Perform enteric coating of step #8 core tablets using step #9 coating dispersion.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A composition for preventing or alleviating a disorder of the gastrointestinal tract wherein the composition comprises at least two active agents selected from a cannabinoid and/or a terpene, wherein one of the active agents is selected from CBD, CBG, CBDA, THC, caryophyllene, limonene, linalool, alpha pinene, myrcene, and humulene.

2. The composition of claim 1, wherein the two active agents act synergistically.

3. A formulation comprising the composition of claim 1 preceding claims.

4. The formulation of claim 3, wherein the formulation is a capsule or tablet.

5. The formulation of claim 4, wherein the formulation is a coated capsule or tablet.

6. The formulation of claim 3, comprising microcyrstalline cellulose, croscarmellose sodium, magnesium stereate, and colloidal silicon dioxide.

7. The formulation of claim 6, further comprising mannitol, and sodium stearyl.

8. The composition of claim 1, wherein the gastrointestinal disorder is selected from Inflammatory bowel syndrome (IBS), gastrointestinal motility disorders; functional gastrointestinal disorders, gastroesophageal reflux disease (GERD), duodenogastric reflux, Crohn's disease, ulcerative colitis, inflammatory bowel disease (IBD), functional heartburn, dyspepsia, functional dyspepsia, nonulcer dyspepsia, gastroparesis, chronic intestinal pseudo-obstruction and colonic pseudo-obstruction.

9. A method for preventing or alleviating a disorder of the gastrointestinal tract, comprising:
a. administering the composition of claim 1 to a subject with a gastrointestinal disorder in a dosage amount sufficient to prevent or alleviate a disorder of the gastrointestinal tract.

10. The method of claim 9, wherein the gastrointestinal disorder is selected from inflammatory bowel syndrome (IBS), gastrointestinal motility disorders; functional gastrointestinal disorders, gastroesophageal reflux disease (GERD), duodenogastric reflux, Crohn's disease, ulcerative colitis, inflammatory bowel disease (TBD), functional heartburn, dyspepsia, functional dyspepsia, nonulcer dyspepsia, gastroparesis, chronic intestinal pseudo-obstruction, and colonic pseudo-obstruction.

11. The method of claim 10, wherein the administration is by oral administration.
